(19) United States
(12) Patent Application Publication
(10) Pub. No.: US 2024/0404303 A1
(43) Pub. Date: Dec. 5, 2024

(54) SYSTEMS AND METHODS FOR LABEL-FREE MULTI-HISTOCHEMICAL VIRTUAL STAINING

(71) Applicant: The Hong Kong University of Science and Technology, Hong Kong (CN)

(72) Inventors: Tsz Wai WONG, Hong Kong (CN); Weixing DAI, Hong Kong (CN); Hei Man WONG, Hong Kong (CN)

(21) Appl. No.: 18/700,782

(22) PCT Filed: Sep. 20, 2022
(86) PCT No.: PCT/CN2022/119857

§ 371 Date: Apr. 12, 2024

Related U.S. Application Data
(60) Provisional application No. 63/254,547, filed on Oct. 12, 2021.

(51) Int. Cl. G06V 20/69; G01N 1/30; G01N 1/31; G06T 7/33; G06V 10/74; G06V 10/774

(52) U.S. Cl. CPC G06V 20/698; G01N 1/30; G01N 1/312; G06T 7/337; G06V 10/761; G06V 10/774; G06V 20/693; G06V 20/695; G01N 2001/302; G06T 2207/10056; G06T 2207/10064; G06T 2207/20081; G06T 2207/30024; G06V 2201/03

(57) ABSTRACT

Systems and methods for multi-spectral and label-free autofluoresence histochemical instant virtual staining are provided. The system includes a sample holder, one or more light sources, an imaging device, and a processor means. The sample holder is configured to secure a sample on a movable structure that is movable in at least two dimensions. The one or more light sources are configured to obliquely illuminate the sample for excitation of the sample. The imaging device is configured to capture images of the sample and the processing means is configured to receive the images of the sample and process the images in accordance with a histochemical virtual staining process. The images include an autofluorescence image and the histochemical virtual staining process includes subdividing the autofluorescence image into a plurality of regions, global sampling a selected region of the autofluorescence image, and classifying the autofluorescence image as one of a plurality of real image classifications or a fake image classification.

### PRIORITY CLAIM

The present application claims priority from U.S. provisional patent application 63/254,547, filed Oct. 12, 2021, the disclosure of which is incorporated by reference herein.

### TECHNICAL FIELD

The present invention generally relates to histochemical staining, and more particularly relates to systems and methods for multi-spectral and label-free autofluorescence histochemical instant virtual staining.

### BACKGROUND OF THE DISCLOSURE

Histological staining chemically introduces a color contrast for the study of specific tissue constituents and is a vital step in diagnosing a wide variety of diseases. While having hematoxylin and eosin (H&E) stain as the most commonly used routine stain for the study of nuclei distribution and cell morphology, other histochemical stains such as special stains or immunohistochemistry (IHC) stains have also been developed to highlight other specific biomolecules and have been in use for over a century. In clinical practice, special stains or IHC stains would occasionally be performed in addition to H&E staining for further confirming of a diagnosis. However, these histochemical stains usually involve irreversible chemical reactions which are destructive to tissue and, therefore, require additional tissue harvesting from patients. Furthermore, the staining process can be time-consuming and labor-intensive, and typically require expensive automated machines to maintain high efficiency.

Alternative stain-free imaging modalities such as stimulated Raman scattering microscopy and non-linear microscopy were initially proposed to replace histochemical staining while generating contrast for studying different molecular features and biomolecules such as collagen. However, since pathologists are well-trained to interpret tissue information based on the color-contrast of histochemically stained images, additional color-mapping is needed after such procedures to generate views analog to histochemical stain. Yet, these pseudo-coloring approaches do not accurately resemble genuine chemically stained images, thus requiring re-training for pathologists (and diagnosis algorithms) to interpret or demanding numerous parameters and fine-tuning to optimize the similarity of such pseudo-coloring to real stains.

Recent advances in deep learning algorithms have inspired many efforts in developing virtual staining solutions to substitute for histological staining aimed at providing significant impacts in speeding up the histopathology workflow; reducing the costs of equipment, reagents, and manpower; and eliminating unnecessary extra tissue harvesting from patients.

Generative Adversarial Network (GAN) is a popular deep learning framework used for virtual staining which is impressive for its ability to generate realistic examples in tasks such as image-to-image translation. GAN framework can be divided into unsupervised and supervised methods depending on whether labeled data is used or not. Several unsupervised methods have been proposed for image-to-image translation such as CycleGAN, CUT, UNIT, and UGATIT which are suitable for virtual staining when labeled data is not available. However, if the structure of training data is complicated, unsupervised methods may fail and the results of virtual staining might not be satisfactory. Compared to unsupervised methods, supervised methods such as pix2pix and other methods can perform better for virtual staining of complicated tissues with labeled training data. Nevertheless, precise image registration is required to ensure that the input images are accurately aligned at the pixel-level. In addition, an identical slide is needed for training to satisfy the stringent registration requirement, which is difficult to obtain and hence poses a restriction on achieving satisfactory transformation via supervised learning.

Since virtual staining is far more efficient than real histochemical staining, especially when multiple staining is needed, generalizing virtual staining to multiple stains would be even more beneficial to the clinical community. To this end, various style transformation approaches have been proposed. For example, an unsupervised method using CycleGAN was proposed to transform real Ki67-CD8 IHC stained images into virtual FAP-CK IHC stained images. Because of the destructive nature of staining, it is challenging to obtain multiple staining results on identical slides. Therefore, precise registration between two staining domains is not possible and limits the use of supervised methods for achieving high accuracy. Later approaches utilize virtual staining results of multiple stains originated from identical slides as learning inputs to achieve perfect registration for supervised learning. However, these approaches rely on common visible features on both input images to ensure high accuracy learning, which poses a type-limitation of stain transformation when biomolecular contrast becomes a constraint.

Thus, there is a need for methods and systems to histochemical virtual staining which overcomes the drawbacks of prior systems and methods and provides time-efficient, inexpensive specific contrast for the study of tissue morphology without requiring undue tissue consumption. Furthermore, other desirable features and characteristics will become apparent from the subsequent detailed description and the appended claims, taken in conjunction with the accompanying drawings and this background of the disclosure.

### SUMMARY

According to at least one aspect of the present embodiments, a system for label-free histochemical virtual staining is provided. The system includes a sample holder, one or more light sources, an imaging device, and a processor means. The sample holder is configured to secure a sample on a movable structure that is movable in at least two dimensions. The one or more light sources are configured to obliquely illuminate the sample for excitation of the sample. The imaging device is configured to capture images of the sample and the processing means is configured to receive the images of the sample and process the images in accordance with a histochemical virtual staining process. The images include an autofluorescence image and the histochemical virtual staining process includes subdividing the autofluorescence image into a plurality of regions, global sampling a selected region of the autofluorescence image, and classifying the autofluorescence image as one of a plurality of real image classifications or a fake image classification.

According to another aspect of the present embodiments, a method for label-free histochemical virtual staining is provided. The method includes subdividing a pair of images of a sample into a plurality of regions, wherein one of the pair of images comprises a first autofluorescence image and a first corresponding image. The method further includes selecting one of the subdivided regions of each of the pair of images, global sampling the selected region of each of the pair of images, and local sampling of portions of the selected region of each of the pair of images, each portion of the selected region of each of the pair of images comprising a multi-pixel cropped patch. Thereafter, the method includes encoding and decoding the locally sampled cropped patch of each of the pair of images to generate a second autofluorescence image and a second corresponding image, and classifying the second autofluorescence image and the second corresponding image as one of a plurality of real image classifications or a fake image classification. The global sampling of the selected region of each of the pair of images includes determining a probability of the selected region being trained in the current iteration in response to a ratio of a similarity index of the selected region to similarity indexes of unselected regions.

### BRIEF DESCRIPTION OF THE DRAWINGS

The accompanying figures, where like reference numerals refer to identical or functionally similar elements throughout the separate views and which together with the detailed description below are incorporated in and form part of the specification, serve to illustrate various embodiments and to explain various principles and advantages in accordance with present embodiments.

FIG. 1 depicts an illustration of a system for label-free histochemical virtual staining in accordance with present embodiments.

FIG. 2 depict a flow diagram for a multi-spectral autofluorescence virtual instant stain (MAVIS) weakly-supervised virtual staining algorithm in accordance with the present embodiments.

FIG. 3, comprising FIGS. 3A to 3E, depicts images of a first region of a human breast biopsy tissue, wherein FIG. 3A depicts an autofluorescence image of the first region of the human breast tissue excited at 265 nm, FIG. 3B depicts an image of hematoxylin and eosin (H&E) virtual staining results achieved by the weakly-supervised method of FIG. 2 in accordance with the present embodiments, FIG. 3C depicts an image of H&E virtual staining results achieved by a conventional unsupervised CycleGAN method, FIG. 3D depicts an image of H&E virtual staining results achieved by a conventional supervised Pix2pix method, and FIG. 3E depicts a real H&E stained ground truth of the autofluorescence image of FIG. 3A.

FIG. 4, comprising FIGS. 4A to 4E, depicts images of a second region of the human breast biopsy tissue depicted in the images of FIGS. 3A to 3E, wherein FIG. 4A depicts an autofluorescence image of the second region of the human breast tissue excited at 265 nm, FIG. 4B depicts an image of hematoxylin and eosin (H&E) virtual staining results achieved by the weakly-supervised method in accordance with the present embodiments, FIG. 4C depicts an image of H&E virtual staining results achieved by a conventional unsupervised CycleGAN method, FIG. 4D depicts an image of H&E virtual staining results achieved by a conventional supervised Pix2pix method, and FIG. 4E depicts a real H&E stained ground truth of the autofluorescence image of FIG. 4A.

FIG. 5, comprising FIGS. 5A to 5D, depicts images of a Schmidtea mediterranea (planaria flat worm), wherein FIG. 5A depicts an image of a low signal-to-noise ratio (SNR) acquisition of the Schmidtea mediterranea, FIG. 5B depicts an image of an output of virtual staining results achieved by a conventional supervised method, FIG. 5C depicts an image of an output of virtual staining results achieved by MAVIS processing in accordance with the present embodiments evidencing denoising in accordance with MAVIS, and FIG. 5D depicts a ground truth image of real staining.

FIG. 6, comprising FIGS. 6A to 6F, depicts images of isotropic reconstruction of a label-stained sample of a developing Danio rerio (zebrafish) eye, wherein FIG. 6A depicts a raw input image of the developing zebrafish eye wherein nuclei were labeled with DRAQ5 magenta staining and nuclei envelopes were labeled with GFP+LAP2B green staining, FIG. 6B depicts a magnified portion of the image of FIG. 6A, FIG. 6C depicts an image of isotropic reconstruction results by a conventional virtual staining supervised method, CARE, FIG. 6D depicts a magnified portion of the image of FIG. 6C, FIG. 6E depicts an image of isotropic reconstruction results by the MAVIS virtual staining method in accordance with the present embodiments, and FIG. 6F depicts a magnified portion of the image of FIG. 6E.

FIG. 7, comprising FIGS. 7A to 7H, depicts images of human lung large-cell carcinoma tissue, wherein FIG. 7A depicts an autofluorescence image of the human lung cancer tissue excited at 265 nm, and FIG. 7B depicts a magnified image of the boxed portion of FIG. 7A, FIG. 7C depicts a H&E-stained MAVIS result of the autofluorescence image of FIG. 7A, FIG. 7D depicts a Masson's Trichrome stained MAVIS result of the autofluorescence image of FIG. 7A, FIG. 7E depicts a H&E-stained MAVIS result of the autofluorescence image of FIG. 7B, FIG. 7F depicts a Masson's Trichrome stained MAVIS result of the autofluorescence image of FIG. 7B, FIG. 7G depicts a real H&E-stained ground truth of the autofluorescence image of FIG. 7B, and FIG. 7H depicts a real Masson's Trichrome stained ground truth of the autofluorescence image of FIG. 7B.

FIG. 8, comprising FIGS. 8A to 8F, depicts images of autofluorescence images of human lung large-cell carcinoma tissue excited by two different light wavelengths, wherein FIGS. 8A and 8B depict autofluorescence images of the human lung large-cell cancer tissue excited by 265 nm, FIGS. 8C and 8D depict autofluorescence images of the human lung large-cell cancer tissue excited by 340 nm, FIG. 8E depicts an image of a real H&E-stained ground truth of the autofluorescence images of FIGS. 8A and 8C, and FIG. 8F depicts an image of a real H&E-stained ground truth of the autofluorescence images of FIGS. 8B and 8D.

FIG. 9, comprising FIGS. 9A to 9E, depicts images of virtual staining results for H&E stain on autofluorescence images of a mouse spleen sample at different excitations, wherein FIG. 9A depicts an autofluorescence image of the mouse spleen sample excited by a 265 nm light emitting diode (LED), FIG. 9B depicts an image of a MAVIS virtual H&E stain result achieved in accordance with the present embodiments of the mouse spleen sample of FIG. 9A, FIG. 9C depicts an autofluorescence image of the mouse spleen sample excited by a 340 nm LED, FIG. 9D depicts an image of a MAVIS virtual H&E stain result achieved in accordance with the present embodiments of the mouse spleen sample of FIG. 9C, and FIG. 9E depicts a real H&E-stained ground truth of the mouse spleen sample.

And FIG. 10, comprising FIGS. 10A to 10E, depicts images of virtual staining results for reticulin stain on autofluorescence images of a mouse spleen sample at different excitations, wherein FIG. 10A depicts an autofluorescence image of the mouse spleen sample excited by a 265 nm LED, FIG. 10B depicts an image of a MAVIS reticulin virtual stain result achieved in accordance with the present embodiments of the mouse spleen sample of FIG. 10A, FIG. 10C depicts an autofluorescence image of the mouse spleen sample excited by a 340 nm LED, FIG. 10D depicts an image of a MAVIS reticulin virtual stain result achieved in accordance with the present embodiments of the mouse spleen sample of FIG. 10C, and FIG. 10E depicts a real reticulin-stained ground truth of the mouse spleen sample.

Skilled artisans will appreciate that elements in the figures are illustrated for simplicity and clarity and have not necessarily been depicted to scale, and that the number in the graphs may have been normalized for simplicity and clarity.

### DETAILED DESCRIPTION

The following detailed description is merely exemplary in nature and is not intended to limit the invention or the application and uses of the invention. Furthermore, there is no intention to be bound by any theory presented in the preceding background of the invention or the following detailed description. It is the intent of present embodiments to present unique systems and methods for multi-virtual staining method, termed multi-spectral autofluorescence virtual instant stain (MAVIS), which include multispectral autofluorescence microscopy and a weakly-supervised virtual staining algorithm and can enable rapid and robust virtual staining of label-free tissue slides to multiple types of histological stains. The multi-spectral imaging systems in accordance with the present embodiments provide a versatile image contrast for highlighting specific biomolecules while a weakly-supervised virtual staining algorithm that does not require pixel-level registration provides improved robustness and accuracy over traditional supervised methods.

Enhanced LED-based multi-spectral imaging techniques are utilized in accordance with the present embodiments to highlight specific biomolecules based on their optical absorption property thereby advantageously providing a better image contrast for virtual staining. Based on autofluorescence contrast, MAVIS is a label-free imaging modality in accordance with the present embodiments that preserve tissues for later analysis. As a wide-field imaging technique, MAVIS does not require any high-repetition-rate laser to maintain high imaging speed as in point scanning techniques (e.g. SRS and non-linear microscopy); therefore MAVIS is also cost-effective since only LEDs are needed.

Prior success of multiple virtual staining has been demonstrated based on brightfield contrast and autofluorescence contrast. Different from conventional multispectral implementations in the visible range, systems and methods in accordance with the present embodiments also incorporate autofluorescence contrast excited in the ultraviolet (UV) light range, thus providing rich endogenous contrast for visualizing multiple biomolecules, e.g. nicotinamide adenine dinucleotide hydride (NADH), collagen and elastin, and similar biomolecules. Since different biomolecules exhibit different absorption properties at different wavelengths, the use of different excitation wavelengths was investigated specifically for different types of virtual stains. Followed by a novel, weakly-supervised algorithm, the difference of virtual staining performance between excitation wavelengths was evaluated to successfully demonstrate the importance of multi-spectral imaging to improve multi-virtual staining. Different from supervised methods, the weakly-supervised algorithm in accordance with the present embodiments does not require identical slides for precise registration. Since such identical slides are clinically hard to obtain, the weakly-supervised algorithm in accordance with the present embodiments provides a higher robustness in training.

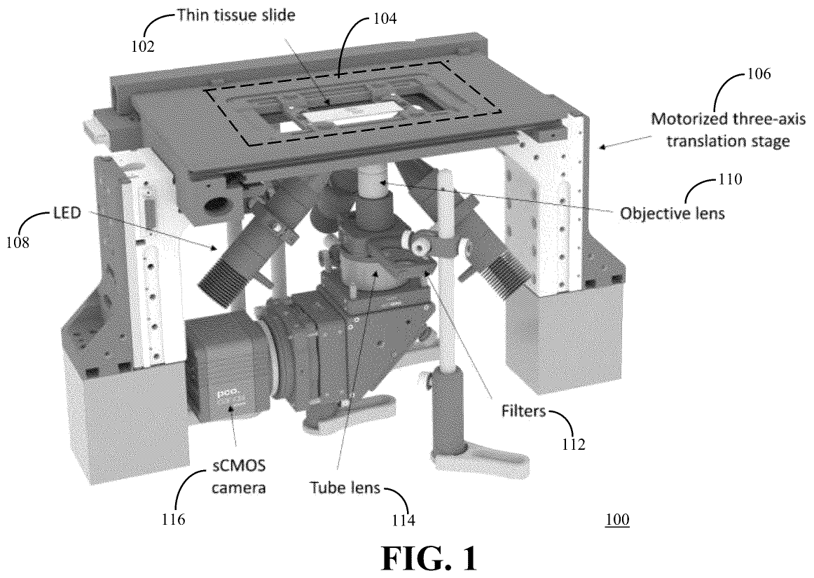

Referring to FIG. 1, a perspective illustration 100 depicts an exemplary multispectral autofluorescence microscopy system for label-free histochemical virtual staining in accordance with present embodiments. A thin formalin-fixed paraffin-embedded slide 102 is placed on a sample holder 104 in conjunction with a XYZ motorized stage 106 (such as a FTP-2000 motorized stage by Applied Scientific Instrumentation). The sample is illuminated obliquely by multiple light-emitting diodes (LEDs) 108 one by one (such as a 265 nm LED M265L4 and a 340 nm LED M340L4 by Thorlabs Inc.). The LEDs 108 are first collimated by an aspherical UV condenser lens with a numerical aperture (NA) of 0.69 (such as a #33-957 lens by Edmund Optics Inc.) and then focused on the sample through a UV Fused Silica Plano-convex lens with a focal length of 50.2 mm (such as a LA4148 lens by Thorlabs Inc.). The excited autofluorescence signal is then collected by an inverted microscope equipped with a 10× objective lens 110 (such as a plan fluorite objective lens with a NA=0.3 by Olympus NDT Inc.), a sliding filter insert (such as a CFS1 filter insert by Thorlabs Inc.) with filters 112 (such as a 450 nm long pass FEL0450 filter by Thorlabs Inc.), an infinity-corrected tube lens 114 (such as a TTL-180-A lens by Thorlabs Inc.), and finally imaged by a monochrome scientific complementary metal-oxide semiconductor (sCMOS) camera 116 (such as a PCO panda 4.2 sCMOS camera having a sensor size 13.3 mm×13.3 mm by PCO AG). After imaging by the sCMOS camera 116, the one or more captured excited autofluorescence images of the sample are stored for later processing by a processing means such as a computer (not shown).

After acquiring the autofluorescence images, the tissue slide is stained with the target stain such as hematoxylin and eosin (H&E), Masson's trichrome or reticulin stain (by Abcam plc.) and then imaged under a whole-slide scanner (such as a 20× NanoZoomer-SQ scanner having a NA=0.75 by Hamamatsu Photonics K.K.) to acquire a stained image for training.

In accordance with the present embodiments, image pre-processing and registration includes first stitching raw autofluorescence images using conventional means such as a grid stitching plugin in ImageJ. The autofluorescence image and the corresponding histochemically-stained image are coarsely registered by estimating the optimal transform based on corresponding points on the two images. An Otsu's method is used to obtain a threshold to estimate the range of background noise values and the number of pixels below the threshold is recorded. Since the distribution of noise should be dominant, an interquartile range method is used to estimate the outlier and define the cut-off value. Pixel values that are equal to or less than the outlier are set to zero. The top 0.1% of the pixel values are also saturated and the remaining pixel values are linearly adjusted.

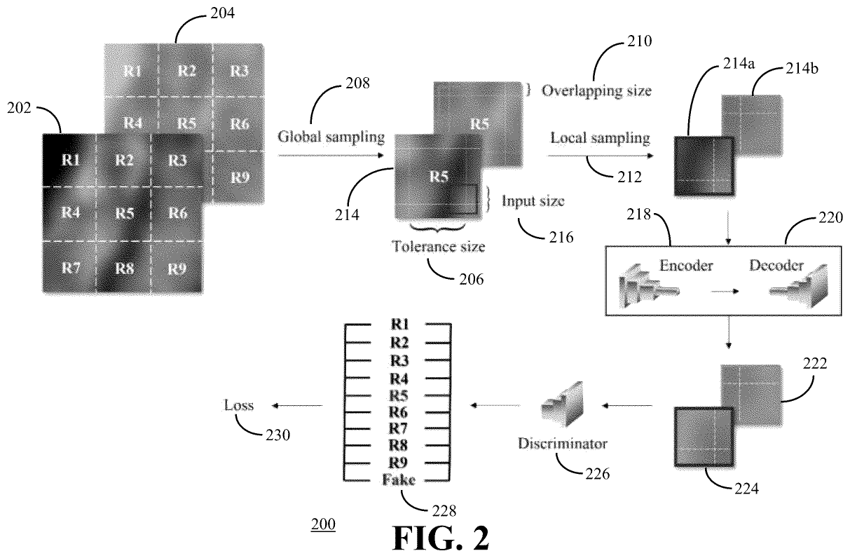

After pre-processing the image, a weakly-supervised virtual staining algorithm in accordance with the present embodiments is utilized for further image processing. Different from typical fully supervised methods, the weakly-supervised virtual staining algorithm in accordance with the present embodiments advantageously only requires patch-level paired images instead of pixel-level paired images to achieve efficient and accurate transformation. The MAVIS weakly-supervised virtual staining algorithm in accordance with the present embodiments is detailed in a flow diagram 200 of FIG. 2 which illustrates a process for generating classes such that one class corresponds to one patch in the raw image and a discriminator 226 is responsible for classification of these classes in accordance with the present embodiments.

Referring to the flow diagram 200, an autofluorescence image 202 and a corresponding H&E image 204 are first simultaneously divided into multiple regions (R1, R2, R3, . . . ) whose length is defined as tolerance size 206. In each iteration, a region (e.g., R5) is selected for global sampling 208 according to the global sampling rule depicted in Equation (1):

$${P}_{i} = \frac{{Q}_{i}}{{\sum }_{j = 1}^{n} {Q}_{j}} \qquad ( 1 )$$

$${Q}_{i} = \frac{1 + \frac{cov ( 255 - AF , HE )}{{σ}_{A F} {σ}_{H E}}}{\sqrt{{\sum }_{k = 0}^{255} {\sum }_{j = 1}^{n} {H}_{i k} {H}_{j k}}} \qquad ( 2 )$$

where Pᵢ is the probability of the selected region being trained in the current iteration; Qᵢ is the similarity index defined by Equation (2); σ_{AF} and σ_{HE} are the standard deviations of the autofluorescence image 202 and the H&E image 204, respectively; cov is the covariance; Hᵢₖ is the number of pixels that have a luminance of k in the selected region i in the autofluorescence image 202 and Hⱼₖ is the number of pixels that have a luminance of k for region j in the autofluorescence image 202 (j=1, . . . , n). Qᵢ consists of a modified Pearson correlation between the flipped autofluorescence image 202 and the H&E image 204 in the numerator such that the higher the similarity between the two domains, the higher the probability of sampling. The denominator is a dot product of pixel distribution between the selected region and the other regions such that the lower the similarity, which indicates the lower the occurrence, the higher the sampling probability should be given to train the rare and special region.

The selected region is expanded by several layers so that it overlaps and shares structures with its neighbor regions, thereby creating an overlapping size 210. For selected regions that reach the edges of images, padding layers of overlapping size are added to create the overlapping size 210.

Next, local sampling 212 is performed by randomly cropping patches 214 from the selected region. With a fixed size of 128×128 (i.e., input size 216), the cropped patches 214a, 214b are then fed into an encoder 218 and a decoder 220 to generate a fake H&E image 222 and a fake autofluorescence image 224.

The model described here is an extension of the traditional GAN architecture, where the discriminator 226 not only tries to classify examples as real or fake classes, but also differentiates different regions in the real class as different classes to improve training accuracy. Therefore, the discriminator 226 should be able to identify N+1 classes 228, including N classes for different regions and an additional class for fake generated examples. A Loss 230 is the cross-entropy loss between the target results 228 of the discriminator 226 and the predicted results of the discriminator 226. The deep neural network architecture of the encoder 218, the decoder 220, and the discriminator 226 in accordance with the present embodiments are listed in TABLE 1, where N is the number of regions that can be potentially selected for training.

TABLE 1

| | | | | | |
|---|---|---|---|---|---|
| Layer | Output size | Kernel size | Stride | Padding | Activation |

Encoder:

| | | | | | |
|---|---|---|---|---|---|
| conv | 256 × 256 × 64 | 7 × 7 | 1 | 3 | Relu |
| conv | 128 × 128 × 128 | 4 × 4 | 2 | 1 | Relu |
| conv | 64 × 64 × 256 | 4 × 4 | 2 | 1 | Relu |
| Resnet × 5 | 64 × 64 × 256 | 3 × 3 | 1 | 1 | Relu |
| | 64 × 64 × 256 | 3 × 3 | 1 | 1 | None |

Decoder:

| | | | | | |
|---|---|---|---|---|---|
| Resnet × 5 | 64 × 64 × 256 | 3 × 3 | 1 | 1 | Relu |
| | 64 × 64 × 256 | 3 × 3 | 1 | 1 | None |
| Upsample | 128 × 128 × 256 | None | None | None | None |
| conv | 128 × 128 × 128 | 5 × 5 | 1 | 2 | Relu |
| Upsample | 256 × 256 × 128 | None | None | None | None |
| conv | 256 × 256 × 64 | 5 × 5 | 1 | 2 | Relu |
| conv | 256 × 256 × 3 | 7 × 7 | 1 | 3 | Tanh |

Discriminator:

| | | | | | |
|---|---|---|---|---|---|
| conv | 128 × 128 × 64 | 4 × 4 | 2 | 1 | Leaky relu |
| conv | 64 × 64 × 128 | 4 × 4 | 2 | 1 | Leaky relu |
| conv | 32 × 32 × 256 | 4 × 4 | 2 | 1 | Leaky relu |
| conv | 16 × 16 × 512 | 4 × 4 | 1 | 1 | Leaky relu |
| conv | 16 × 16 × | 1 × 1 | 1 | 0 | None |
| | (N + 1)* | | | | |

The virtual staining performance of MAVIS in accordance with the present embodiments was compared with conventional supervised and unsupervised methods and the results of such comparison are depicted and discussed hereinafter.

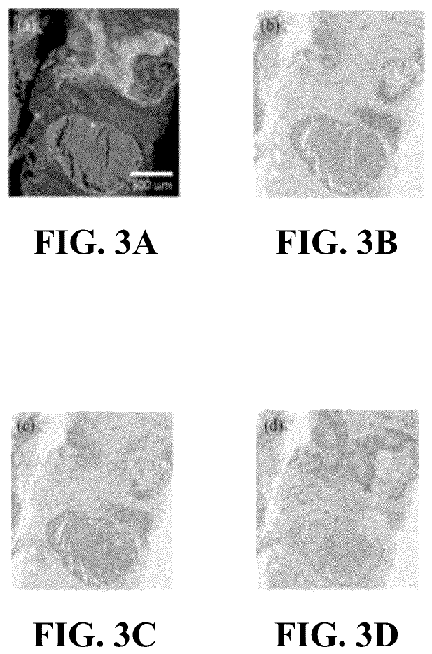

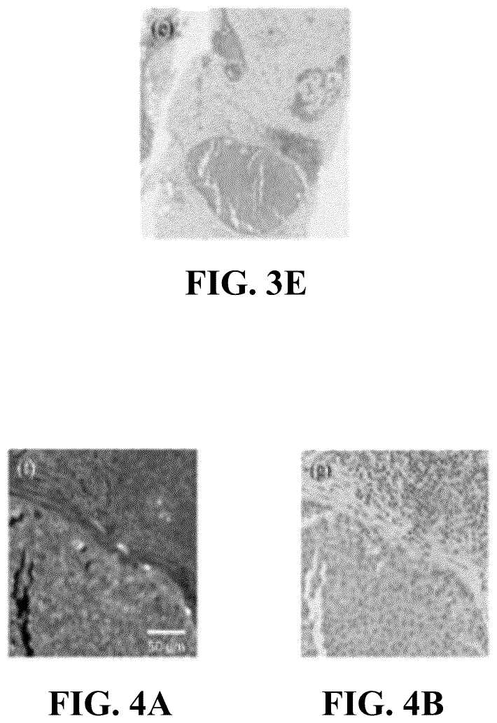

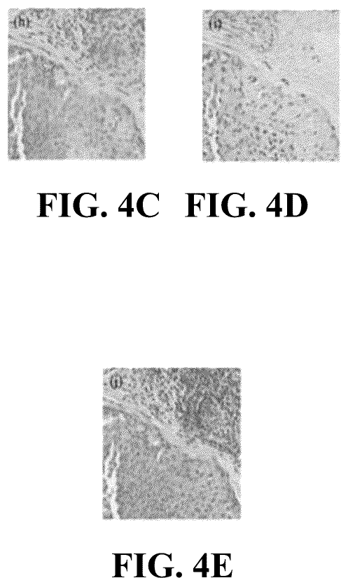

Referring to FIGS. 3A to 3E and FIGS. 4A to 4E, a human breast biopsy tissue was virtually stained and compared with unsupervised (CycleGAN) and supervised (Pix2pix) methods to evaluate the performance of MAVIS.

FIG. 3A depicts a first region of an autofluorescence image of a human breast biopsy tissue excited at 265 nm. FIGS. 3B, 3C and 3D depict images of H&E virtual staining results of the autofluorescence image of FIG. 3A achieved by the weakly-supervised method in accordance with the present embodiments (FIG. 3B), a conventional supervised method (FIG. 3C), and a conventional unsupervised method (FIG. 3D). And FIG. 3E depicts an image a real H&E-stained ground truth of the autofluorescence image of FIG. 3A.

FIG. 4A depicts a second region of the autofluorescence image of the human breast biopsy tissue excited at 265 nm different from the first region in the autofluorescence image of FIG. 3A. FIGS. 4B, 4C and 4D depict images of H&E virtual staining results of the autofluorescence image of FIG. 4A achieved by the weakly-supervised method in accordance with the present embodiments (FIG. 4B), a conventional supervised method (FIG. 4C), and a conventional unsupervised method (FIG. 4D). And FIG. 4E depicts an image a real H&E-stained ground truth of the autofluorescence image of FIG. 4A.

Given the same image data size, the MAVIS images of FIGS. 3B and 4B not only show superior performance over the unsupervised output which failed to transform the complicated tissue morphology shown in the images of FIGS. 3D and 4D, but also excitingly outperform the supervised method shown in the images of FIGS. 3C and 4C with fewer artifacts generated.

To quantitively compare the performance of MAVIS with unsupervised and supervised methods, Fréchet inception distance (FID) is used to quantitively measure the statistical difference between the virtually stained and the real H&E image where the smaller the distance, the higher the similarity. In addition, Multi-scale Structural Similarity (MS-SSIM) is also used to compute the overall similarity with a weighted evaluation at different resolution and scale. These quantitative measurements of different virtual staining methods for the human breast biopsy tissue are summarized in TABLE 2. From TABLE 2, it is clear that the MAVIS output advantageously evidences the smallest FID and the highest MS-SSIM values which agree with the visual perception in FIGS. 3B and 4B and support the better performance achieved by MAVIS when compared with the unsupervised and supervised methods.

TABLE 2

| Method | CycleGAN | Pix2pix | MAVIS |
|---|---|---|---|
| FID | 28.4131 | 25.9485 | 18.9612 |
| MS-SSIM | 0.5625 | 0.6100 | 0.6172 |

To further evaluate the performance of MAVIS algorithm, we also demonstrated the applicability of MAVIS in image restoration such as denoising and isotropic reconstruction.

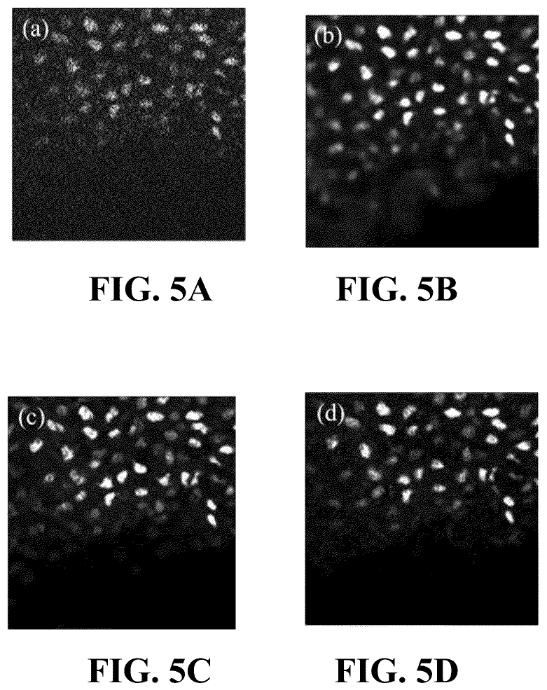

FIGS. 5A to 5D depict images of a Schmidtea mediterranea (planaria flat worm) comparing denoising of the MAVIS method in accordance with the present embodiments to denoising of conventional supervised virtual staining methods. FIG. 5A depicts an image of a low signal-to-noise ratio (SNR) acquisition of the Schmidtea mediterranea. FIG. 5B depicts an image of an output of virtual staining results achieved by a conventional supervised method while FIG. 5C depicts an image of an output of virtual staining results achieved by MAVIS processing in accordance with the present embodiments evidencing denoising in accordance with MAVIS. For comparison, FIG. 5D depicts a ground truth image of real staining.

For image denoising on the planaria dataset shown in the FIGS. 5A to 5D, the output of MAVIS in FIG. 5C has an FID of 37.3689 and a MS-SSIM of 0.7426, which results in closer image quality to the ground truth shown in FIG. 5D than that of the supervised method called CARE shown in FIG. 5B which has a FID of 152.4581 and a MS-SSIM of 0.6969. In addition, the supervised method generates some fake nuclei in the background as seen in FIG. 5B, while this same region for MAVIS as shown in FIG. 5C was correct and closer to the ground truth shown in FIG. 5D.

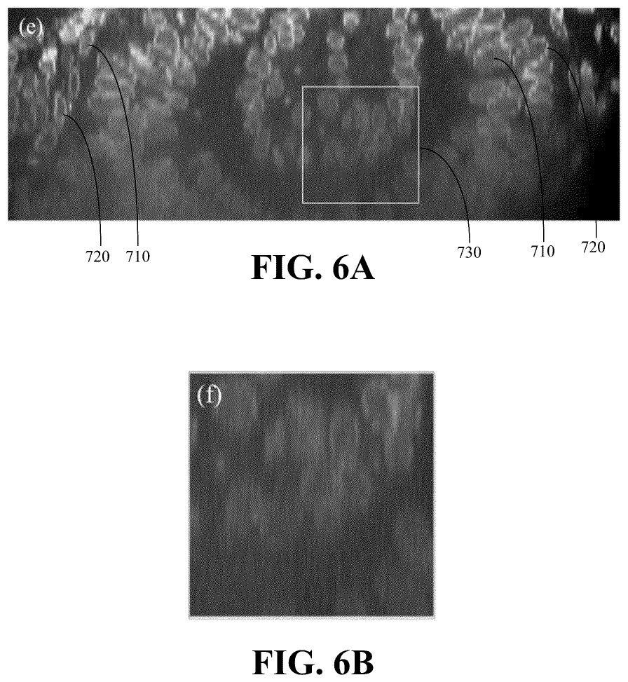

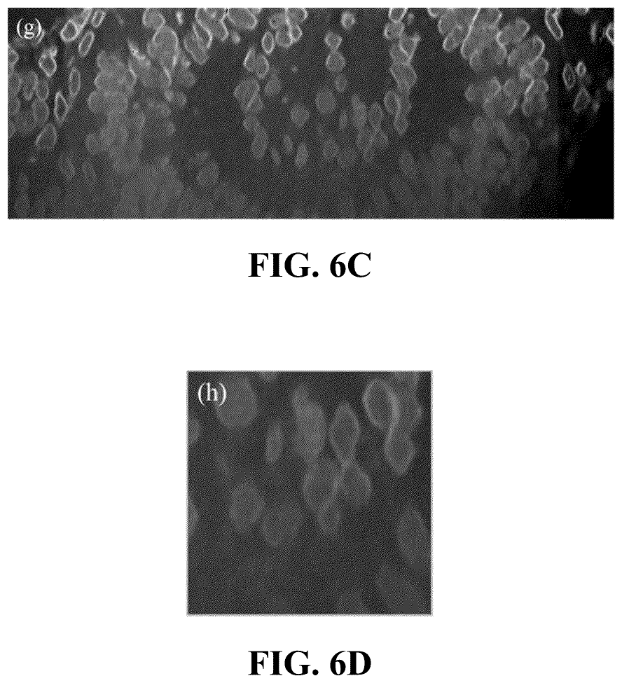

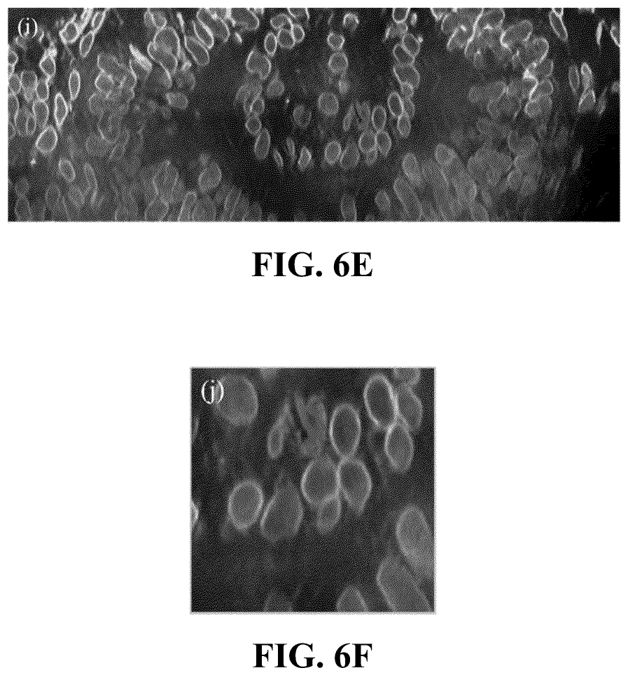

Referring to FIGS. 6A to 6F, images depict isotropic reconstruction of a label-stained sample of a developing Danio rerio (zebrafish) eye. FIG. 6A depicts a raw input image of the developing zebrafish eye wherein nuclei 710 were labeled with DRAQ5 magenta staining and nuclei envelopes 720 were labeled with GFP+LAP2B green staining and FIG. 6B depicts a magnified view of a portion 630 of the image of FIG. 6A. FIG. 6C depicts an image of isotropic reconstruction results by the conventional virtual staining supervised method, CARE, with FIG. 6D depicting a corresponding magnified portion. FIG. 6E depicts an image of isotropic reconstruction results by the MAVIS virtual staining method in accordance with the present embodiments, and FIG. 6F depicts its corresponding magnified portion.

For the zebrafish eye dataset shown in FIGS. 6A to 6F, isotropic restoration was applied for both the supervised method (FIGS. 6C, 6D) and the MAVIS method (FIG. 6E, 6F). Compared to the supervised method, MAVIS generates a clearer image with a sharper green nuclear envelope in the GFP+LAP2B channel which should normally be found at the edge surrounding the magenta nuclei, especially in the bottom restoration region. However, only the magenta nuclei were clearly restored while the envelope can be barely seen in the bottom region generated by supervised method.

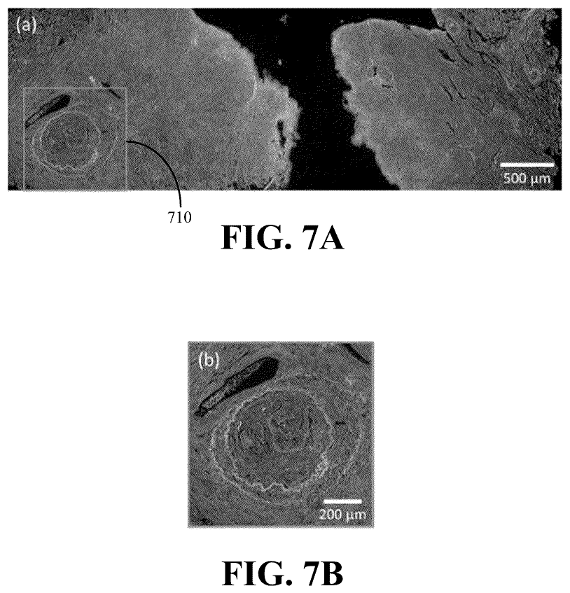

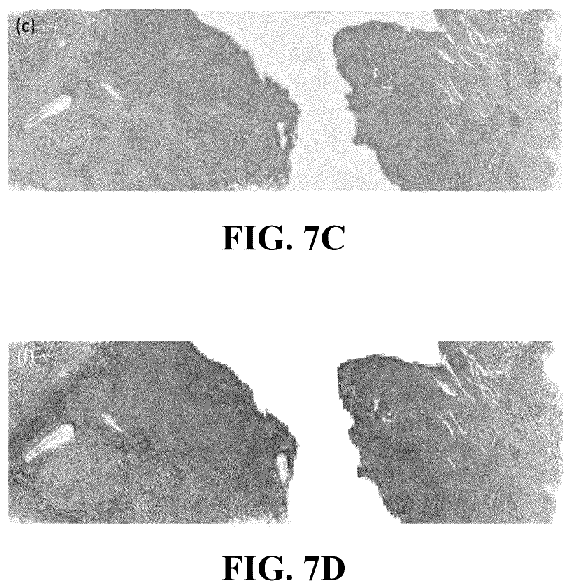

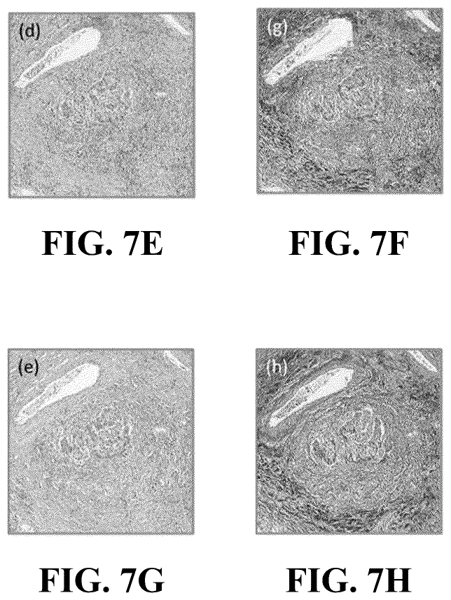

The performance of the novel MAVIS algorithm was examined on other stains and organs. Referring to FIGS. 7A to 7H, images of human lung cancer tissue are depicted. As illustrated in FIG. 7A, the human lung cancer tissue excited at 265 nm was imaged. FIG. 7B depicts a zoomed in autofluorescence image of the boxed portion 710 of the image of FIG. 7A. In order to examine the multi-virtual staining performance of the MAVIS method in accordance with the present embodiments, both H&E staining and Masson's Trichrome staining was performed on adjacent portions of the autofluorescence image to acquire the training data for H&E stain and the training data for Masson's Trichrome stain. Masson's Trichrome is commonly used for highlighting type I collagen. FIGS. 7C and 7E depict the H&E-stained MAVIS result and the Masson's Trichrome stained MAVIS result of the autofluorescence image of FIG. 7A, respectively, while FIGS. 7D and 7F depict the H&E-stained MAVIS result and the Masson's Trichrome stained MAVIS result of the autofluorescence image of FIG. 7B, respectively. For comparison, FIGS. 7E and 7H depict a real H&E-stained ground truth and a real Masson's Trichrome stained ground truth, respectively, of the autofluorescence image of FIG. 7B.

As noted above, the training set for Masson's Trichrome is not originated from the identical slide as the training set for H&E but originated on adjacent slides. Even though not identical slides, MAVIS still can achieve reasonable staining output for H&E (FIG. 7F) that is highly similar to the adjacent reference slide stained by Masson's Trichrome (FIG. 7H). This also shows the robustness of MAVIS to be trained on an adjacent slice, which normally compromises the accuracy in conventional supervised virtual staining methods.

To investigate the use of different excitation wavelengths specifically for different types of virtual stains and the contrast difference introduced by the different excitation wavelengths, autofluorescence images were obtained at two excitation wavelengths, 265 nm and 340 nm. Since the absorption of DNA and RNA peak at ˜265 nm which also coincides with an absorption peak of NADH which is one of the key fluorophores in human tissue (Em 460 nm), a 265 nm excitation wavelength should naturally form an intrinsic negative contrast between dark nuclei and bright cytoplasm, which can correlate with a nuclei contrast in H&E stain.

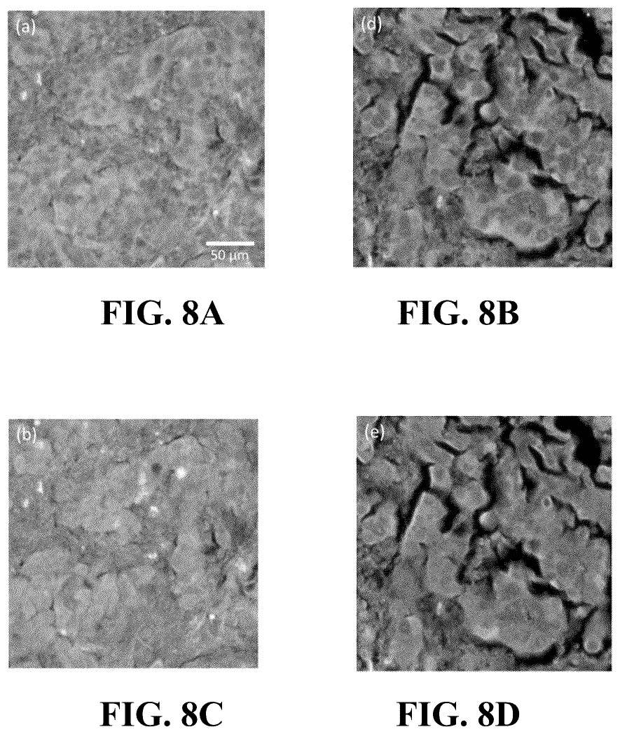

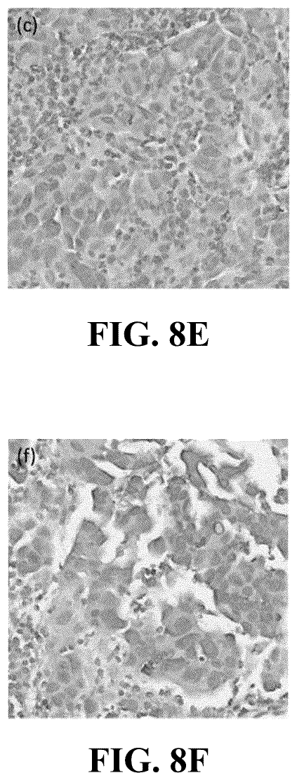

FIGS. 8A and 8B depict autofluorescence images of a human lung large-cell cancer tissue excited by 265 nm, while FIGS. 8C and 8D depict autofluorescence images of the human lung large-cell cancer tissue excited by 340 nm. FIG. 8E depicts an image of a real H&E-stained ground truth of the autofluorescence images of FIGS. 8A and 8C, and FIG. 8F depicts an image of a real H&E-stained ground truth of the autofluorescence images of FIGS. 8B and 8D. The better nuclei contrast shown in the images of 265 nm excitation than the images of 340 nm excitation is likely due to lower absorption of both DNA, RNA, and NADH at 340 nm.

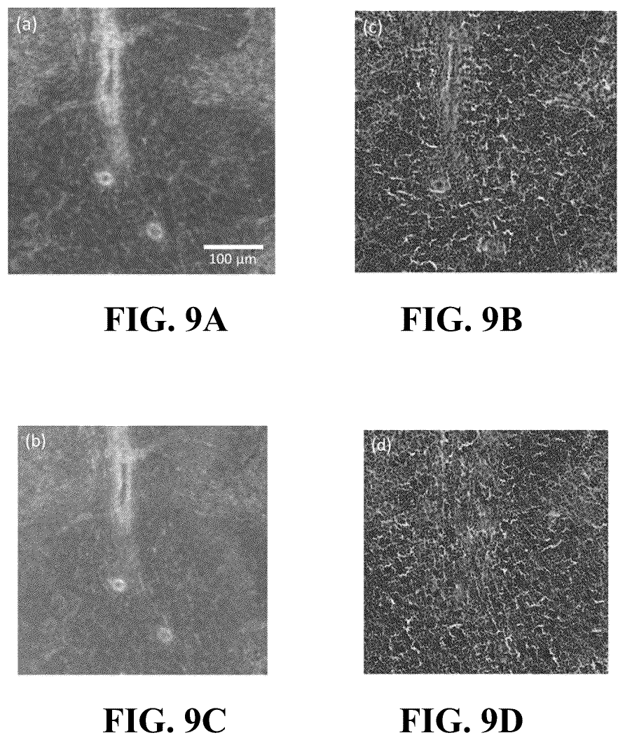

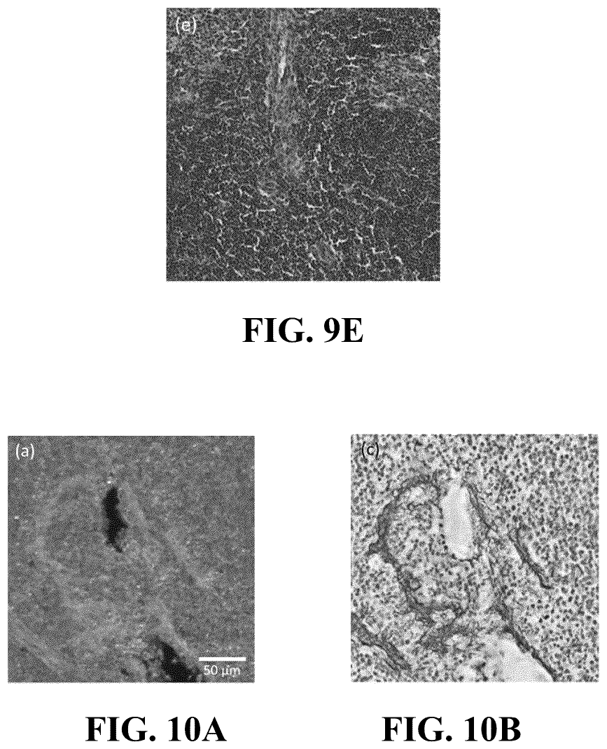

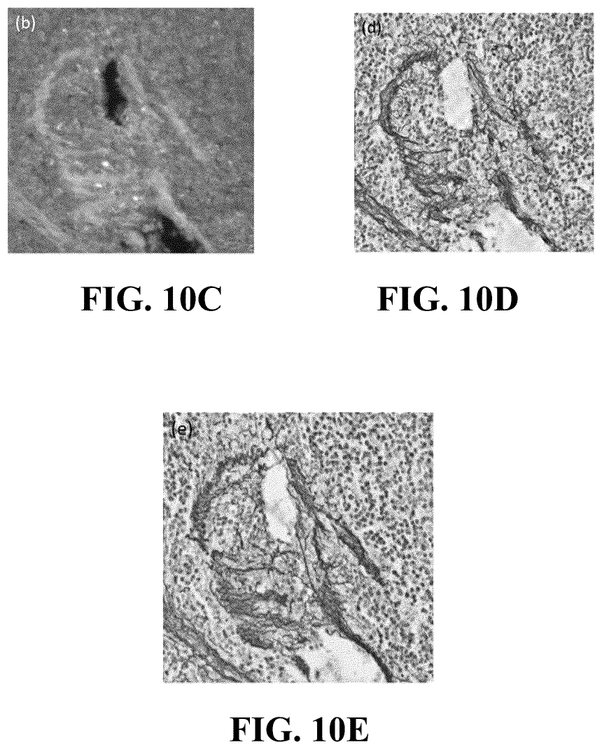

Virtual staining performance of different excitation wavelengths was examined by comparing the virtual staining results for H&E stain and reticulin stain based on the autofluorescence images excited by 265 nm and 340 nm, respectively. FIG. 9A depicts an autofluorescence image of a mouse spleen sample excited by a 265 nm LED and FIG. 9B depicts an image of a MAVIS virtual stain result achieved in accordance with the present embodiments of the mouse spleen sample of FIG. 9A. Similarly, FIG. 9C depicts an autofluorescence image of the mouse spleen sample excited by a 340 nm LED and FIG. 9D depicts an image of a MAVIS virtual stain result achieved in accordance with the present embodiments of the mouse spleen sample of FIG. 9C. FIG. 9E depicts a real H&E-stained ground truth of the mouse spleen sample for comparison. The virtual staining results in FIGS. 9B and 9D show a better H&E transformation based on the autofluorescence image excited by 265 nm (FID of 19.1618, MS-SSIM of 0.5371) than the image excited by 340 nm (FID of 25.3847, MS-SSIM of 0.5453).

Reticulin fibers, composed of collagen type III, are abundant in the spleen which serves as a supportive framework to its cellular constituents. FIG. 10A depicts an autofluorescence image of a mouse spleen sample excited by a 265 nm LED and FIG. 10B depicts an image of a MAVIS reticulin virtual stain result achieved in accordance with the present embodiments of the mouse spleen sample of FIG. 10A. Similarly, FIG. 10C depicts an autofluorescence image of the mouse spleen sample excited by a 340 nm LED and FIG. 10D depicts an image of a MAVIS reticulin virtual stain result achieved in accordance with the present embodiments of the mouse spleen sample of FIG. 10C. FIG. 10E depicts a real reticulin-stained ground truth of the mouse spleen sample for comparison.

Reticulin fibers cannot be shown in H&E stain but can be stained black by silver in reticulin stain. Since collagen has higher absorption than NADH at 340 nm and has a broad emission range around 380 nm, thus providing explanation for the excellent collagen contrast excited by 340 nm as shown in FIGS. 10C and 10D. The virtual staining result from the 340 nm autofluorescence image of FIG. 3D (FID of 39.7752, MSSSIM of 0.5497) also shows a better performance than the 265 nm autofluorescence image of FIG. 10B (FID of 48.3618, MSSSIM of 0.5241), which missed some reticulin fibers when compared with the ground truth of FIG. 10E.

Thus, it can be seen that the methods and systems in accordance with the present embodiments provide a novel and efficient multi-spectral autofluorescence virtual instant stain method called MAVIS to achieve virtual staining of multiple histological stains. The weakly-supervised MAVIS algorithm in accordance with the present embodiments advantageously does not require pixel-level registration as in supervised method while only patch-level paired data is needed for training, significantly improving the robustness while preserving the capability of learning complicated features. The exemplary results shown and described hereinabove prove that the MAVIS systems and methods can achieve even higher similarity to the ground truth than the conventional fully supervised systems and methods. Also, exemplary results shown and described hereinabove demonstrate an excitation wavelength that highlights specific biomolecules can improve the virtual staining performance, thereby showing that the multispectral imaging system in accordance with the present embodiments has great potential for providing versatile contrast for transforming different histological stains.

While exemplary embodiments have been presented in the foregoing detailed description of the present embodiments, it should be appreciated that a vast number of variations exist. It should further be appreciated that the exemplary embodiments are only examples, and are not intended to limit the scope, applicability, operation, or configuration of the invention in any way. Rather, the foregoing detailed description will provide those skilled in the art with a convenient road map for implementing exemplary embodiments of the invention, it being understood that various changes may be made in the function and arrangement of steps and method of operation described in the exemplary embodiments without departing from the scope of the invention as set forth in the appended claims.

What is claimed is:

1. A method for label-free autofluorescence histochemical instant virtual staining comprising:
subdividing a pair of images of a sample into a plurality of regions, wherein one of the pair of images comprises a first autofluorescence image and a first corresponding image;
selecting one of the subdivided regions of each of the pair of images;
global sampling the selected region of each of the pair of images;
local sampling of portions of the selected region of each of the pair of images, each portion of the selected region of each of the pair of images comprising a multi-pixel cropped patch;
encoding and decoding the locally sampled cropped patch of each of the pair of images to generate a second autofluorescence image and a second corresponding image; and
classifying the second autofluorescence image and the second corresponding image as one of a plurality of real image classifications or a fake image classification, wherein global sampling of the selected region of each of the pair of images comprises determining a probability of the selected region being trained in the current iteration in response to a ratio of a similarity index of the selected region to similarity indexes of unselected regions.

2. The method in accordance with claim 1 further comprising imaging the sample to derive the first autofluorescence image and the first corresponding image.

3. The method in accordance with claim 2 wherein imaging the sample comprises excitation of the sample at a predetermined light frequency to generate the first autofluorescence image.

4. The method in accordance with claim 1 further comprising staining the sample to generate the first corresponding image comprising a histochemically-stained image.

5. The method in accordance with claim 4 wherein staining the sample comprises staining the sample in accordance with a target stain selected from the group comprising a hematoxylin and eosin stain, a Masson's trichrome stain and a reticulin stain.

6. The method in accordance with claim 1 further comprising label-free histochemical instant virtual staining of a further autofluorescence image in response to the plurality of real image classifications and the fake image classification.

7. The method in accordance with claim 1 further comprising after global sampling the selected region of each of the pair of images, expanding a size of the selected region of each of the pair of images.

8. The method in accordance with claim 7 wherein expanding the size of the selected region of each of the pair of images comprises overlapping the selected region of each of the pair of images with portions of neighboring regions of the selected region.

9. The method in accordance with claim 1 further comprising coarsely registering the first autofluorescence image and the first corresponding image by estimating an optimal transform based on corresponding points on the first autofluorescence image and the first corresponding image before subdividing the first autofluorescence image and the first corresponding image.

10. The method in accordance with claim 1 wherein global sampling of the selected region of each of the pair of images further comprises determining the similarity index of the selected region in response to number of pixels having particular luminances.

11. A system for label-free histochemical virtual staining comprising:
a sample holder configured to secure a sample on a movable structure, the movable structure movable in at least two dimensions;
one or more light sources configured to obliquely illuminate the sample for excitation thereof;
an imaging device configured to capture images of the sample; and
a processing means configured to receive the images of the sample and process the images in accordance with a histochemical virtual staining process, wherein the images comprise an autofluorescence image, and wherein the histochemical virtual staining process comprises subdividing the autofluorescence image into a plurality of regions, global sampling a selected region of the autofluorescence image, and classifying the autofluorescence image as one of a plurality of real image classifications or a fake image classification.

12. The system in accordance with claim 11 wherein the one or more light sources comprises at least one light emitting diode, each of the at least one light emitting diode configured to output light of a predetermined frequency for excitation of the sample in accordance with the predetermined frequency.

13. The system in accordance with claim 11 wherein the processing means is further configured to train a weakly-supervised virtual training method for the histochemical virtual staining process.

14. The system in accordance with claim 13 wherein the images comprise a pair of images comprising the autofluorescence image and a corresponding stained image, and wherein the processing means is configured to train the weakly-supervised virtual training method by global sampling a selected region of each of the pair of images, wherein global sampling the selected region of each of the pair of images comprises determining a probability of the selected region being trained in the current iteration in response to a ratio of a similarity index of the selected region to similarity indexes of unselected regions.
